(19) United States
(12) Patent Application Publication
(10) Pub. No.: US 2009/0246073 A1
(43) Pub. Date: Oct. 1, 2009

(54) Apparatus and method for inline solid, semisolid, or liquid antimicrobial treatment

(76) Inventor: Rong Yan Murphy, Fayetteville, AR (US)

Correspondence Address: HENRY LAW FIRM, P.O. BOX 8850, FAYETTEVILLE, AR, 72703, US

(21) Appl. No.: 12/056,176

(22) Filed: Mar. 26, 2008

(51) Int. Cl. A61L 2/02; A61L 2/10; A23L 3/26; A61L 2/025

(52) U.S. Cl. 422/20; 422/24; 422/22

(57) ABSTRACT

An antimicrobial treatment method for non-thermal pasteurization and anti-microbial treatment of solid, semisolid, or liquid foods in industrial food transport systems is provided. For solid food applications, the method and related apparatus comprises a conveyor-based transport system. For semisolid or liquid food applications, the method and related apparatus comprises a conduit-based transport system. Both the conveyor-based and conduit-based transport systems are capable of treating food with ultrasound, a UV-ruby lightwave combination, a pulsed electric field, and/or a magnetic field. The method is capable of realizing greater than 3 log reductions in live microbes in foodstuffs, although the technology may be used in nonfood applications.

### CROSS REFERENCES

None.

### GOVERNMENTAL RIGHTS

None.

### FIELD OF INVENTION

This invention pertains to the use of ultrasound, lightwave combinations, pulsed electric fields, or magnetism to treat solids, semisolids, or liquids, including but not limited to foodstuffs, before, during, or after processing or packaging to reduce or neutralize contaminants and/or microbes associated therewith.

### BACKGROUND OF THE INVENTION

Undercooked or contaminated foodstuff has caused illness since ancient times. Today, a wide variety of food processing techniques are used to reduce the risk of food-borne illness, and these techniques include the time-honored methods of heating, toxic inhibition (smoking, pickling, etc.), dehydration, low temperature inactivation (freezing) in addition to more modern techniques such as oxidation, osmotic inhibition (use of syrups), freeze drying, vacuum packing, canning, bottling, jellying, heat pasteurization, and irradiation. Generally, such processes do not actually sterilize food, as full sterilization adversely affects the taste and quality of final foodstuffs, but instead reduce microbial content and inhibit further microbial growth. Despite the numerous processes available to food manufacturers to reduce microbes in food, the risk of food-borne illness continues and thus remains the focus of continuing research and development.

Although generally preventable, food-borne illness remains a serious problem to food consumers, government, and industry. Over one-quarter of the population of the United States is affected every year by food-borne illnesses; contaminated food has been estimated by the World Health Organization to cause 76 million illnesses in the U.S. each year, including 325,000 cases resulting in hospitalization and 5,000 deaths. In many cases, microbial contamination occurs during handling when preparing food for retail sale. Although sanitation policies have been improving during recent years, it has proven very difficult to eliminate contamination and pathogens associated with preparing, handling, and processing food at an industrial level. For example, Listeria monocytogenes cannot be eliminated from food or food processing environments using present technologies. A survey by USDA-FSIS showed that between 1% and 10% of retail ready-to-eat deli foods were contaminated with L. monocytogenes. The potential contamination of these and other microbes in foodstuff processing environments presents a serious and continuing food safety threat, which has promoted interest in applying non-heat treatment to foods that kills bacteria and preserves food characteristics. Treating cut fruits and vegetables, seafood, cheese, deli food, meat, poultry, and other foodstuff with non-heat antimicrobial alternatives can reduce or eliminate the presence of microbes.

It is known that more preventative approaches to food safety can reduce or eliminate physical, chemical, and biological hazards in food. “Hazard Analysis and Critical Control Points” (“HACCP”) is a systematic approach to the handling, preparation, and storage of food that aims to prevent food-borne illness at its source rather than inspecting finished products. HACCP works by identifying the steps at which contamination of food is known to occur, and then controlling the environment surrounding food products during those steps, i.e., preventing the entry of contaminants into the sealed processing environment. HACCP is not a process to treat contaminated product; rather, HACCP is a testing methodology to ensure that each step in the process is free from contaminants as well as a strict recording system to verify the results. It is an object of the invention to reduce or eliminate food-borne microbes at virtually any or all stages of an industrial foodstuff processing or preparation system.

The prior art in the field of treating industrial foodstuff to minimize or reduce microbes and contaminants varies widely in form and function, but most references report results measured as the reduction of microbial content between two or more assays in units of “logs,” which represents the difference in microbial content between the two assays in terms of orders of magnitude. For example, a commonly sought after and reported goal in the prior art is a reduction by 3 log, which means that the microbial content in a particular sample was reduced by 3 orders of magnitude to 0.1% of its original content. It is thus an object of the invention to utilize an industry standard measurement of effectiveness and to likewise provide for at least a 3 log reduction in microbial content.

Perhaps the oldest approach to eliminating harmful microbes from food is by application of substantial heat. In order to apply sufficient heat, modern industrial processes may use ovens which require batch processing. Other industrial processes may use infrared ovens placed inline within standardized foodstuff preparation processes; these infrared ovens focus substantial heat energy directly at the foodstuff while it is being processed, rather than heating a larger enclosed space. To address the contamination or microbial activity, high heat infrared ovens have been used to kill microbes, even on precooked food immediately prior to packaging. The relatively long wavelength of infrared (“IR”) allows IR to penetrate below the food surface; however, IR also produces substantial heat directly on the surface of the foodstuff, which may negatively impact the desired qualities of food, such as color, taste, and texture. For instance, U.S. Pat. No. 6,780,488, issued to Howard, discloses the use of a specific type of infrared oven in which heating elements surround an inline conveyor and reheat precooked food to 500° C. or more. Again, the problem with reheating precooked food to such high temperatures is that such food invariably continues to cook. Changes to taste and color at such temperatures occur and the outer surface of foodstuff such as meat turns tough and brown. Thus, while infrared may be beneficial in some industrial cooking applications, IR is not particularly well-suited to reheating precooked food to treat it against contamination immediately prior to being packaged. IR is even more detrimental to foodstuffs including fruits, vegetables, or other items that have low tolerances to heat. It is thus an object of the invention to meet or exceed the sanitary achievements surrounding the use of IR while also avoiding the high heat that accompanies the use of IR. It is also an object of the invention to avoid the requirement that the foodstuff be processed in batches but rather to permit the foodstuff to remain part of a continuous processing system.

Another technology utilized in the prior art to eliminate microbes and harmful effects of contamination on foodstuffs is ionizing radiation, or irradiation. Irradiation works by ionizing atoms and molecules, i.e., stripping an electron from the orbits of such atoms and molecules, and is typically performed by subjecting foodstuffs to short wavelength, high energy radiation such as x-rays or gamma rays. Not only does irradiation destroy living tissue, it is also believed to create damaging secondary effects through the chemical activity of liberated free radicals. The concern surrounding the effects of irradiation on microorganisms in food remains a subject of debate despite the fact that the side effects of irradiation have been studied for over sixty years. Many persons skilled in the art of irradiation are critical of the irradiation process and claim that irradiation creates new chemicals in food that are not naturally present and do not form as a result of cooking or other processing methods. Additionally, irradiation is thought to adversely affect the taste of food. Public mistrust of irradiation (often called cold pasteurization to reduce the negative connotation associated with the word radiation), slow adoption by the food industry, and conflicting reports as to the safety of this process dictate the need to look to another process. It is thus an objection of the invention to safely eliminate microbes from food without the negative social stigmas associated with ionizing radiation.

The social concern for ionizing radiation may stem from the fact that x-rays and gamma rays naturally occur only at miniscule levels within the earth's atmosphere. In order to avoid the negative consumer perceptions associated with x-ray and gamma ray irradiation, the industry has generally turned to relatively lower-energy, longer wavelength electromagnetic radiation, including ultraviolet light. Ultraviolet light, or UV, is a naturally occurring wavelength of light produced by the sun that partially penetrates the earth's atmosphere in sufficient quantities to have noticeable effects, such as the burning of human skin without heat over a relatively prolonged period of several minutes to a few hours. Similarly, UV is known in the prior art as an effective tool for decreasing the number of living microbes on the surface of some foodstuffs. However, as noted in U.S. Pat. No. 4,396,582 issued to Kodera et al., a known problem with UV is that for it to be effective as an anti-microbial agent, the food surface must have direct contact with UV treatment. Because UV is incapable of appreciable penetration into food products, it is also not a good tool to reduce microbes located anywhere but the surface of the food product. Further, if any packaging or other material is located between the UV source and the food surface, the effectiveness of UV on the food surface is greatly reduced. It is therefore an object of the invention to address the historical inadequacies associated with using UV as an antimicrobial treatment for industrial foodstuff, especially for food products that have already been processed and packaged and are being readied for sale.

Another technology in the field of treating foodstuffs to reduce microbes associated with industrial food processing is ultrasonic treatment, known also as ultrasonication and ultrasound. The prior art suggests that sound waves between about 16 kHz and 100 kHz can be used to eliminate microbes in food and liquids through microbial and enzyme inactivation; however, these suggestions are not well established as such range of ultrasound wavelength has inferior bacterial kill rates. As discussed by U.S. Pat. No. 5,879,732 issued to Caracciolo et al., ultrasound, even at frequencies up to 850 kHz, merely operates to dislodge microbes from food surfaces through the phenomenon of cavitation. Due to the failure of ultrasound in prior art ranges to directly kill or degrade the actual microbes, the prior art teaches away from the use of ultrasound alone as an antimicrobial. Rather, ultrasound is understood to be effective only when used in combination with other antimicrobial agents, such as the antimicrobial agent ozone in Caracciolo, to achieve appropriate kill rates of up to 3 log. Standard industry practice dictates that other antimicrobial treatments must be used in conjunction with ultrasound to produce effective results, and industry literature posits that such results are produced because ultrasound is believed to expose more surface area of the microbes to the complementary antimicrobial agents. It is thus an object of the invention to have the option to utilize ultrasound, alone, but at power and frequency levels beyond those previously used and which will be sufficient to kill microbes at reduction rates exceeding 3 log. It is also an object of the invention to exploit ultrasound for a secondary benefit to the food processing system, i.e., to detect the presence of nonfood material in or on the processed foodstuff.

Electricity is another technology that is known to kill microbes in foodstuff in limited circumstances. According to one sampling of prior art, the application of an electrical field to a liquid solution, as between an anode and cathode, realigns ions within the solution and within the microbes themselves for an antimicrobial effect. This concept, as applied in the food service industry, is generally known as pulsed electric field (“PEF”). According to the prior art, PEF works in some limited liquid applications by destroying or damaging the cellular structure of microbes within the electrical field. Little prior art exists relative to using PEF to treat solid food; for instance, U.S. Pat. No. 5,549,041 (the '041 patent), issued to Zhang et al., discloses a PEF treatment system capable of treating solid food. The '041 patent teaches batch treatment; in order to treat solid food, a container must be removed from the apparatus, completely filled with solid food, and reinstalled on the PEF machine. Such batch process is a disadvantage for industrial food processing, and it is therefore an object of the invention to provide inline PEF antimicrobial treatment of solid foodstuffs.

The prior art reveals that electrical fields used in PEF antimicrobial food treatment range in magnitude between a few hundred volts per centimeter up to 500 kV/cm. One criticism of the use of PEF in the food service industry is that prior art embodiments are not capable of handling the large flow throughput generally necessary for industrial food processing, as the prior art utilized batch treatment or small quantities of food material rather than inline processing of substantial volumes of foodstuff. It is thus an object of the invention to provide a food processing system capable of utilizing an industrial strength PEF system that can effectively treat a substantial quantity of semisolid or liquid food material flowing at 100 gallons per minute or more with at least a 3 log reduction in microbes.

Another technology, similar to PEF, is the use of strong magnetic fields to physically disrupt or rupture certain cellular components and microbes. Little prior art is available on the efficacy of continuous magnetic fields for antimicrobial treatment; some prior art studies analyzed the effect of much smaller magnetic fields over extended time periods, but the purposes of such studies was not to analyze the antimicrobial effect of magnetic fields. While the prior art suggests the use of magnetic fields as a means of inducing a PEF, the prior art actually teaches away from linking magnetic fields with antimicrobial effect. It is thus an object of the invention to provide a foodstuff treatment process that may preferentially utilize strong magnetic fields to eliminate or reduce the presence of microbes in food products, and it is a further object of the invention to benefit public health by eliminating or reducing microbes from both food and nonfood materials.

It is a further object of the invention to disclose new methods of non-thermal or low-thermal anti-microbial treatment that hold significant promise for reducing or eliminating microbes from solid, semisolid, and liquid materials.

Deficiencies of sterilization techniques plague other industries as well, particularly the medical field. Accordingly, it is a further object of the invention to apply to industries in which sterilized items, whether solid, semisolid, or liquid materials, are desirable.

The process, as well as the apparatus in accordance with the invention, provides reliable and relatively inexpensive non-thermal pasteurization and anti-microbial treatment of solid, semisolid, and liquid materials.

### BRIEF SUMMARY OF THE INVENTION

This multi-faceted antimicrobial treatment process and related apparatus solves many different problems of microbial contaminations in solids, semisolids, and liquid materials. The processes and apparatuses of this invention can be used with solid, semisolid, and liquid materials before, during, or after processing or packaging. Specifically, the invention comprises an array of inline antimicrobial devices (IAMDs) designed to utilize at least one of the following forms of energy: (1) ultrasound, (2) lightwave combinations, (3) PEF, or (4) magnetic energy, or a combination thereof.

Generally, inline manufacturing processing of solid food involves a series of conveyors that transport solid foodstuff at a predetermined velocity and inter-spacing to allow for adequate inspection and packaging. Solid foodstuff processing continues uninterrupted until such time as it is desirable to treat the foodstuff with an antimicrobial treatment; in prior art applications using heat, for instance, the inline conveyor system may need to have been interrupted to apply batch antimicrobial heat treatments in an oven. In contrast to the prior art, the invention is useful for inline solid food conveyor systems in that the invention contemplates antimicrobial treatment as a component of the inline conveyor system rather than a separate, batch-type component. Elements of the disclosed invention may be incorporated into one apparatus, or any one of the various forms of energy may be incorporated into distinct, modular apparatuses for placement at different locations throughout the manufacturing process.

The use of ultrasound energy in connection with the invention contemplates at least two possible embodiments for the treatment of (1) solid food and (2) semisolid or liquid food. Both of these embodiments subject foodstuff to ultrasound energy levels substantially higher than previously utilized or disclosed by others in the field; as noted earlier, ultrasound below about 1 MHz does not kill microbes, rather it has been suggested to merely dislodge microbes from the surface of food. The invention utilizes ultrasound at a frequency between about 100 kHz to 2 MHz, and preferably between about 1 MHz to 2 MHz. In the higher portion of this frequency range, the ultrasound destroys or substantially disrupts the cellular integrity of relevant microbes. When ultrasound technology used in either solid or semisolid/liquid applications is coupled with a sensing means consistent with this disclosure, the end result is to also alert the user to the existence of nonfood particles in the foodstuff.

The use of lightwave energy applies to the treatment of (1) solid food and (2) semisolid or liquid food. Both of these embodiments subject microbes to novel combinations of lightwaves, i.e., the combination of visible red light (“ruby light”) and UV light in order to achieve a proper reduction in the microbe population of a given foodstuff. The lightwave embodiments of the invention use UV light having a wavelength between about 10 nanometers (nm) and 400 nm and ruby light at a wavelength between about 560 nm and 1,000 nm. The various lightwave combination embodiments contemplated by this invention harness constructive interference. This phenomenon occurs when the two different wavelengths of light interfere, and the result is substantially deeper penetration of the short-wavelength UV light beyond the food surface and into the actual foodstuff. While the lightwave combination component of this invention proves effective under conditions where the UV and ruby light combination is applied to food alone, such embodiment is particularly effective over the prior art when used in connection with prepackaged foodstuffs. That is, the combination of ruby light with UV light energy overcomes the limitations associated with the use of UV light alone, by penetrating many forms of plastic packaging. This advance is even more effective than the prior art at penetrating dark plastic packaging. Further, by using ruby light rather than infrared radiation, the radiated heat generated by the process is much lower, thereby decreasing the amount of heat applied to the food in furtherance of one object of the invention.

The use of PEF energy applies to the treatment of (1) solid food and (2) semisolid or liquid food. The PEF energy components of the invention contemplate a unique electrode design that permits treatment of a substantial volume of food. The PEF electrodes must communicate with materials with sufficient conductivity, such as metal conduit or water vapor, to create an electric field of sufficient magnitude to effectively treat the foodstuff. Whereas prior art designs used to treat solid food required batch processing of containers entirely filled with solid food to solve this goal, the invention preferentially relies instead upon ionized air, water vapor, or steam to provide a conductive medium between the electric field and solid food during inline processing. As to semisolid/liquid food applications, prior art PEF designs relied upon relatively large electrodes (as compared to the diameter of the conduit) that were embedded into spans of straight conduit, thereby impeding laminar flow. In contrast, the invention does not rely upon electrodes embedded within the conduit but rather comprises electrodes that either communicate with the conduit or form at least a portion of the conduit itself. The placement of the electrodes, the conductive medium used in solid food applications, and the large flow requirements that necessitate conduits with larger diameters in semisolid/liquid food applications require PEF of a magnitude higher than is disclosed or suggested in the prior art; the PEF embodiments have fields with energies between about 2 kV/cm and 2,000 kV/cm, and preferably between about 500 kV/cm and 2,000 kV/cm. Optionally, the use of a spiraled conduit, rather than a straight conduit, in conjunction with the PEF embodiment allows substantially more pulses from a smaller PEF unit than would be feasible in a straight pipe configuration, thereby adding to the effectiveness of PEF treatment according to the invention.

The magnetic energy component of the invention contemplates the use of magnets to create high strength magnetic fields that disrupt or destroy the integrity of vital intracellular processes and structures of microbes in solid or semisolid/liquid foodstuffs. The magnetic embodiment that treats solid food comprises a nonmagnetic conveyor transversely configured with a magnetic coil. The magnetic embodiment that treats semisolid/liquid food comprises a conduit constructed of substantially nonmagnetic material transversely surrounded by a magnetic coil. For both solid and semisolid/liquid treatment, the magnetic embodiments are capable of applying a continuous magnetic field having a strength between 1 and 20 Teslas. The preferred placement of the magnetic coil perpendicular to the flow of foodstuff permits the foodstuff to be subjected to the most intense portion of the magnetic field, while at the same time allowing for the magnetic treatment apparatus to be confined to a relatively small space. Optionally, the use of a spiraled conduit, rather than a straight conduit, in conjunction with the semisolid/liquid magnetic embodiment allows substantially more magnetic force from a smaller unit than would be feasible in a straight pipe configuration.

### BRIEF DESCRIPTION OF THE DRAWINGS

FIG. 1 is a perspective view of a conveyor system used for antimicrobial treatment of solid food according to various embodiments of the invention.

FIG. 2 is a perspective view of a conduit system used for antimicrobial treatment of semisolid or liquid food according to various embodiments of the invention.

FIG. 3 is a perspective view of the apparatus associated with the ultrasound energy treatment method as applied to solid foodstuff.

FIG. 4 is a perspective view of the semisolid or liquid food application of the ultrasound energy treatment method of the invention.

FIG. 5 is a perspective view of the solid food lightwave embodiment of the invention.

FIG. 6 is a perspective view of the semisolid or liquid food lightwave embodiment of the invention.

FIG. 7 is a perspective view of a device used to generate ruby light in the lightwave embodiments of the invention.

FIG. 8 is a perspective view of the solid food PEF embodiment of the invention.

FIG. 9 is a perspective view of a preferred configuration of the semisolid/liquid PEF embodiment of the invention using regular straight conduit.

FIG. 10 is a cross-section view of a preferred configuration for the semisolid/liquid PEF embodiment of the invention wherein the electrodes and insulator form the smooth walls of the conduit.

FIG. 11 is a perspective view of a preferred configuration of the semisolid/liquid PEF embodiment of the invention wherein the conduit is coiled.

FIG. 12 is a perspective view of another preferred configuration for the semisolid/liquid PEF embodiment of the invention wherein the conduit is coiled.

FIG. 13 is a perspective view of the solid food magnetic embodiment of the invention.

FIG. 14 is a perspective view of the semisolid or liquid food magnetic embodiment of the invention.

These and other advantages of the invention will become apparent from the following detailed description which, when viewed in light of the accompanying drawings, discloses the embodiments of the invention.

### LISTING OF COMPONENTS

101—conveyor IAMD
103—conveyor
105—food transport system
107—conduit IAMD
109—conduit
111—coil
113—housing jacket
115—ultrasound driver
117—UV light
119—ruby light
121—transparent window
123—filament
125—housing
127—glass
129—opaque material
131—pane
133—anode
135—cathode
137—insulator
139—magnetic coil

### DETAILED DESCRIPTION OF THE INVENTION

The preferred embodiments of the invention are designed for industrial food processing facilities but may be used in conjunction with standardized processing of other materials and products other than foodstuff.

Typical food processing facilities utilize food transport systems to move foodstuffs along various processing stations so that the foodstuff may be continually processed from raw goods to finished and/or packaged products. Food transport systems are often conveyor-based for solid or prepackaged foods, pipe-based for semisolid or liquid foods, or a combination of both. In connection with industrial processing, the foodstuff may be contaminated by microbes that may be inherent to the foodstuff or is a residue of the processing system. Food processing systems are generally prone to microbial growth.

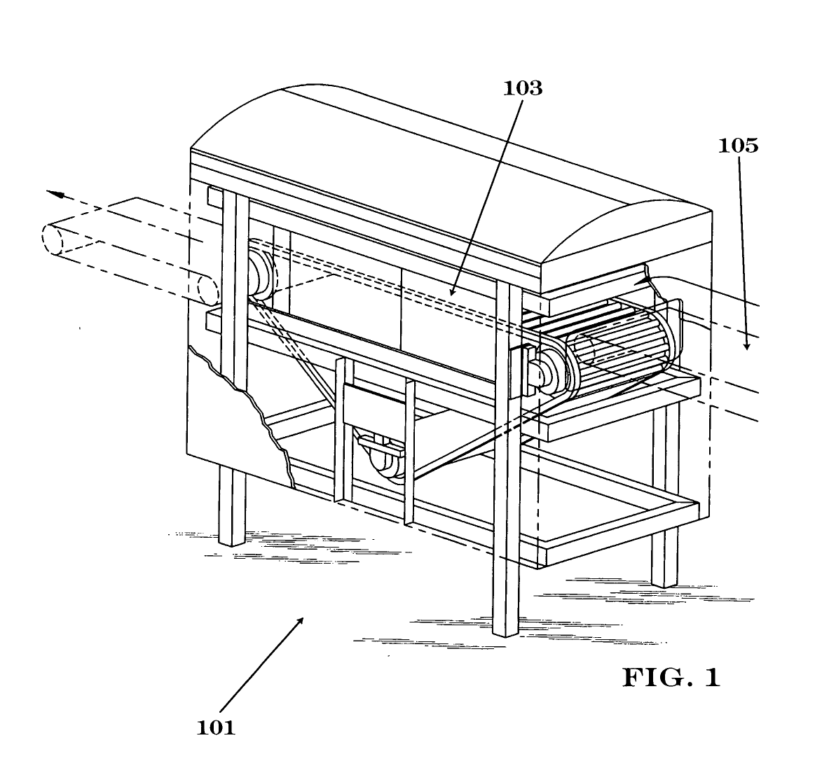

FIG. 1 represents an inline processing station for solid foodstuff, which generally comprises a conveyor IAMD 101 through which foodstuff from a food transport system travels, subjects the foodstuff to antimicrobial treatment, and permits the foodstuff to be transported uninterrupted through the continuous food transport system. Conveyor IAMD 101 may utilize a conveyor belt 103 to transport solid or prepackaged food. In this solid food context, food transport system 105 is depicted as a generic foodstuff delivery system that deposits foodstuff onto conveyor belt 103. Conveyor belt 103 thereafter transports foodstuff to and through conveyor IAMD 101. In typical industrial situations, foodstuff can move along conveyor at approximately 120 pounds per minute (lbs/min), and conveyor IAMD 101 is constructed to handle foodstuff moving at up to 150 lbs/min. The conveyor IAMD 101 houses a means of antimicrobial treatment using one of four energy systems disclosed in more detail, infra.

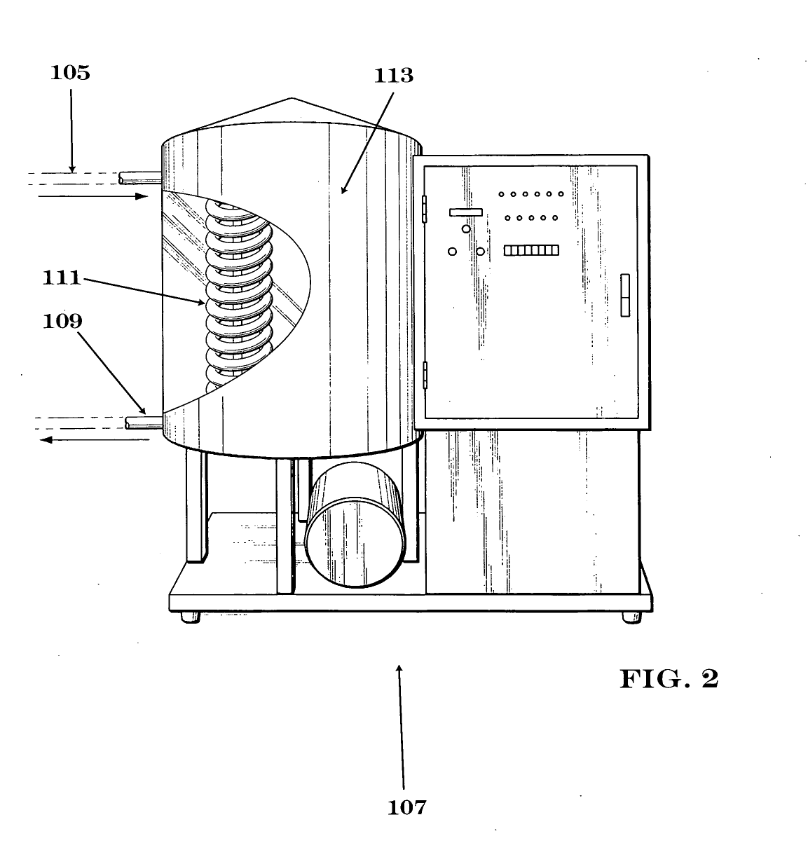

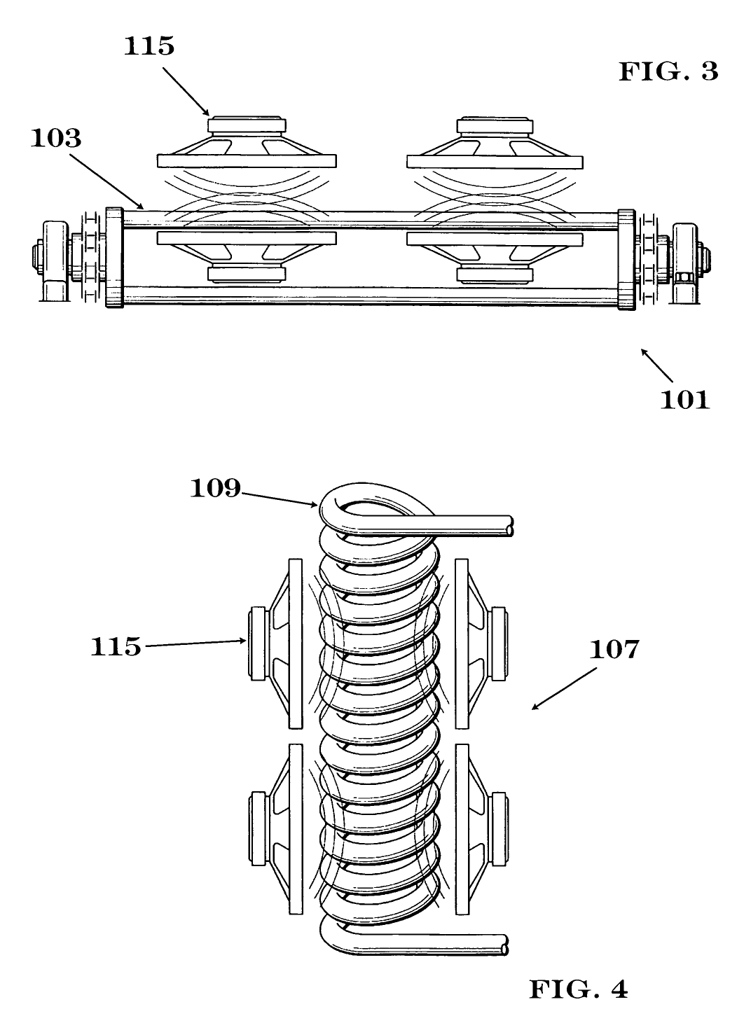

FIG. 2 represents an inline processing station for semisolid or liquid foodstuff, which generally comprises conduit IAMD 107 that utilizes a transport conduit 109 for semisolid or liquid foodstuff. Food transport system 105, in this semisolid/liquid context, provides sufficient foodstuff into transport conduit 109, and the pressurization provided by food transport system 105 ensures sufficient foodstuff is supplied through conduit IAMD 107. Antimicrobial treatment using one of four energy systems disclosed in more detail, infra, is delivered to transport conduit 109.

To facilitate efficient use of any of the four energy systems in the semisolid/liquid application, the transport conduit 109 may be spiraled or shaped into series of coils 111. In such instance, a housing jacket 113 may substantially surround coils 111. While the flow rate of industrial liquid food transport systems can vary widely, for example, from 1 gallon to 300 gallons per minute, typical industrial low-viscosity semisolid or liquid food transport systems have a flow rate requirement of not less than 100 gallons per minute through 1.5 inch conduit, or 385 cubic inches per second through conduit having a cross-sectional area of 1.77 square inches. The flow rate for higher viscosity semisolid foodstuff will depend upon the demands of the given application.

As example to illustrate the benefit of a coiled application, assume the velocity of the foodstuff in semisolid or liquid food processing systems is 218 inches per second (100 gallons per minute). By coiling 13.8 turns of 1.5 inch conduit inside a 5 inch diameter coil, the width required to contain a 218-inch length of conduit drops to 20.7 inches, or less than one-tenth the distance of a corresponding volumetric analysis of straight pipe. Optionally, the diameter of the conduit can be moderately increased (without increasing the diameter of the coil) to address concerns of reduced flow associated with turbulence due to the curved conduit. The coil configuration disclosed herein thus conserves valuable processing plant floor space and increases residency time in the antimicrobial kill zone afforded by one of four energy systems disclosed in more detail, infra.

The invention comprises seven distinct preferred embodiments, as follows: (1) ultrasound energy for antimicrobial treatment of solid foodstuff; (2) ultrasound energy for antimicrobial treatment of semisolid or liquid foodstuff; (3) lightwave energy for antimicrobial treatment of solid foodstuff; (4) lightwave energy for antimicrobial treatment of semisolid or liquid foodstuff; (5) PEF energy for antimicrobial treatment of solid foodstuff; (6) PEF energy for antimicrobial treatment of semisolid or liquid foodstuff, (7) magnetic energy for antimicrobial treatment of solid foodstuff; and (8) magnetic energy for antimicrobial treatment of semisolid or liquid foodstuff.

### Ultrasound Energy

The first preferred embodiment uses ultrasound energy for the antimicrobial treatment of solid foodstuff. Referring to FIG. 3, the solid foodstuff ultrasound embodiment is a conveyor IAMD 101 that has one or more ultrasound drivers 115 mounted above, below, or surrounding conveyor 103. Foodstuff travels on conveyor 103, and ultrasound drivers 115 emit ultrasound waves directed towards foodstuff traveling on conveyor 103, thereby delivering an effective antimicrobial treatment. The measure of an effective amount of ultrasound energy will depend upon the rate of travel of the solid foodstuff and the duration of exposure to such energy.

The second preferred embodiment uses ultrasound energy for the antimicrobial treatment of semisolid or liquid foodstuff. The second preferred embodiment is a conduit IAMD 107 having one or more ultrasound drivers 115 associated with conduit 109. Ultrasound drivers 115 produce ultrasound waves that penetrate food in conduit 109. The ultrasound waves produced by ultrasound drivers 115 are directed towards conduit 109 in a direction substantially normal to the surface of conduit 109.

In the first and second preferred embodiments, ultrasound drivers 115 receive an electric signal from an integrated circuit or amplifier (not shown) that cause the ultrasound drivers 115 to produce ultrasound having a wavelength of between about 100 kilohertz (kHz) and 2 megahertz (MHz), and preferably between about 855 kHz and 2 MHz. At sufficient power, between about 1 kilowatt (kW) to 10 kW of power per kilogram (kg) of food, ultrasound at this frequency causes microbial cell lysis. The duration of exposure is calculated according to the specific application, but effective is deemed to be at least a 3 log reduction in the microbial content.

Optionally, ultrasound drivers 115 can be supplemented with ultrasound transducers. Ultrasound transducers are capable of receiving an ultrasound wave reflected by the food; if other nonfood material is in or on food, then it returns a different, typically brighter ultrasound signature than food. Ultrasound transducers can be used for quality control purposes; the signals returned by the ultrasound transducers can be fed into programmable logic controllers (“PLC” or “PLCs”) to recognize and thereafter divert food contaminated with nonfood particles out of the food processing system. The ultrasound transducer signals can also be fed into an electronic monitoring system to determine the amount of nonfood material present within the food processing system to ensure that the levels of nonfood material comply with FDA and other regulatory or sanitary requirements. For example, in meat processing systems, the ultrasound transducer signals may alert the presence of bone fragments, metal, plastic, or any other foreign material having a temperature, salinity, or moisture content substantially different from the foodstuff being subjected to the ultrasound treatment.

### Lightwave Energy

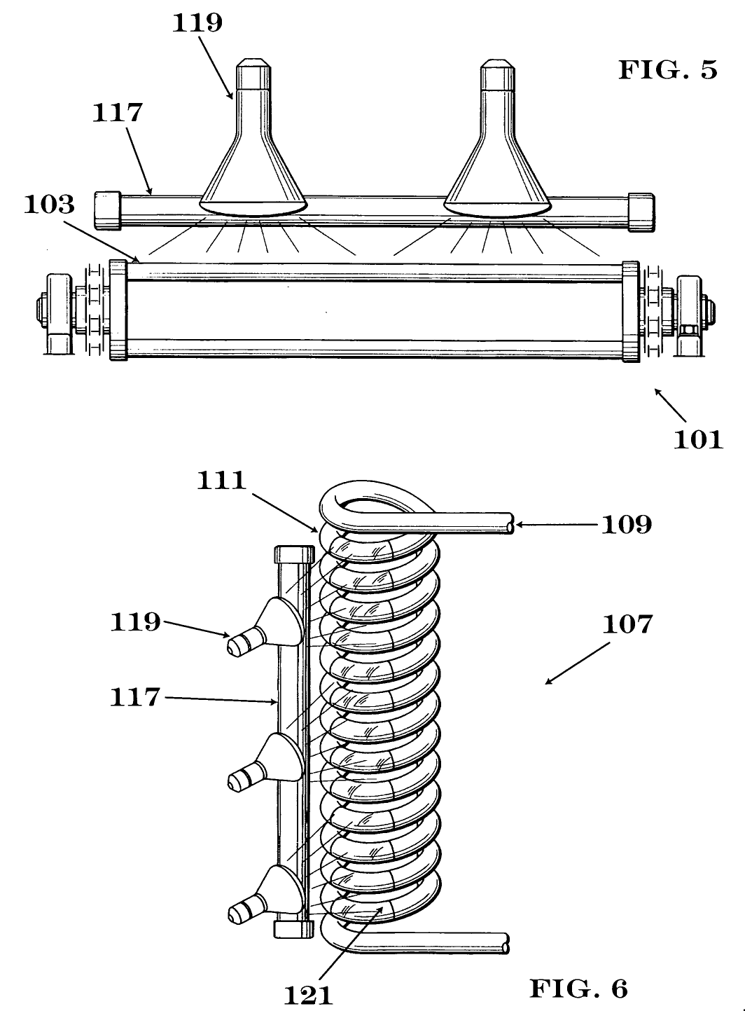

The third preferred embodiment uses lightwave energy for the antimicrobial treatment of solid foodstuff. Referring now to FIG. 5, the third preferred embodiment is a conveyor IAMD 101 that has one or more UV lights 117 mounted above, below, or surrounding conveyor 103. One or more ruby lights 119 is also mounted above, below, or surrounding conveyor 103. Foodstuff traveling along conveyor 103 is effectively bathed in the combination of UV and ruby light. Preferably, the third preferred embodiment ensures that sufficient surface of foodstuff traveling on conveyor 103 receives a sufficient amount UV and ruby light to effectively treat against microbes. Further to this goal, conveyor 103 may rotate food as it travels through conveyor IAMD 101. Conveyor 103 may also be constructed of a material or designed in a way that minimizes interference with or scattering of UV light to ensure an effective treatment of the foodstuff with UV and ruby light while the foodstuff is traveling along conveyor 103. For example, conveyor 103 may preferably be made of fine stainless steel mesh, quartz glass rods, or a transparent plastic mesh.

The fourth preferred embodiment uses lightwave energy for the antimicrobial treatment of semisolid and liquid foodstuff. Referring now to FIG. 6, the fourth preferred embodiment has one or more UV lights 117 and one or more ruby lights 119 mounted transversely around conduit 109. For straight conduit, UV lights 117 and corresponding ruby lights 119 will effectively bathe conduit 109. If conduits are configured in coil 111, UV lights 117 and ruby lights 119 may be mounted either around the exterior of coil 111 or may also be mounted within the interior of coil 111, or both. In order to allow effective absorption of UV and ruby light, at least a portion of conduit 109 is constructed of transparent material 121, and is preferably constructed of high-quality silica or quartz glass, which can be transparent to all UV wavelengths and which results in low attenuation losses for ruby light.

In the third and fourth preferred embodiments, food is simultaneously exposed to UV light between about 10 nanometers (nm) and 400 nm and ruby light at a wavelength between about 560 nm and 1,000 nm. UV light at such wavelengths, and particularly at wavelengths at about 250 to 260 nm, inactivates microbes. When used in conjunction with UV light, ruby light assists UV light in penetrating food packaging and the surface of food products.

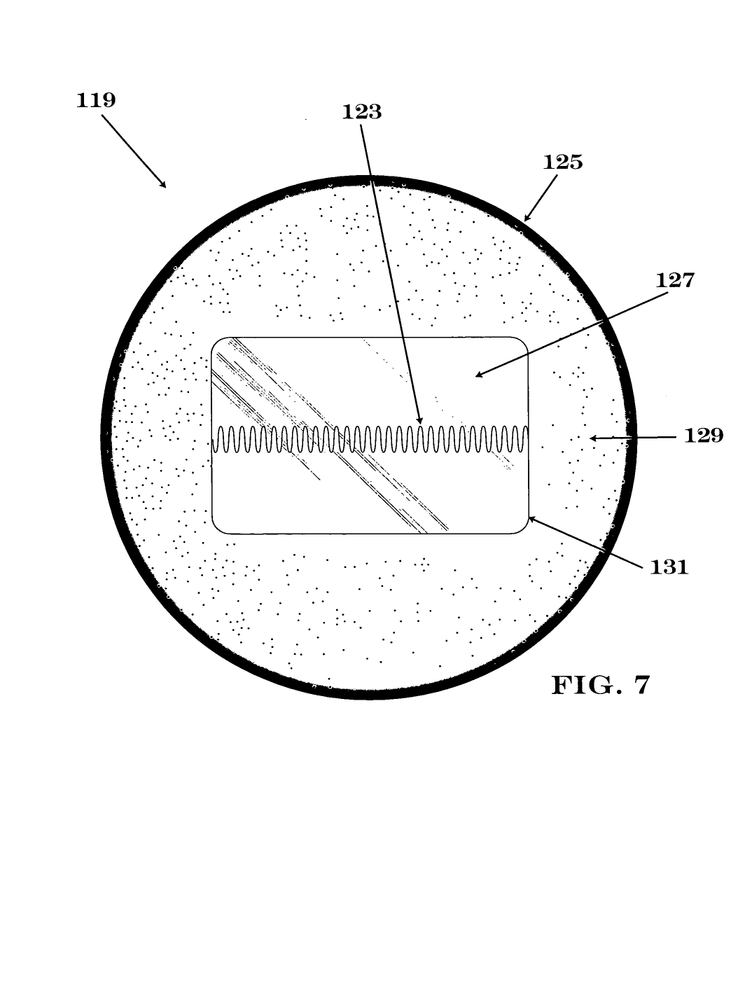

Referring now to FIG. 7, one or more ruby light 119 may also be constructed by enclosing a filament 123 in a housing 125 closed on one side by a piece of glass 127. Glass 127 is made of high quality silica or quartz glass. Preferably, glass 127 is plated with opaque material 129 that permits control over the direction and intensity of ruby light to form pane 131. Even more preferably, opaque material also has reflective properties so that light striking opaque material 129 is reflected back into housing 125 until it exits pane 131, which substantially eliminates the problem of wasted energy due to absorption of light by opaque material 129. While the ruby light 119 is shown in FIGS. 5-7 as having a shape similar to light bulbs of the prior art, no such limitation is intended.

### Pulsed Electric Field Energy

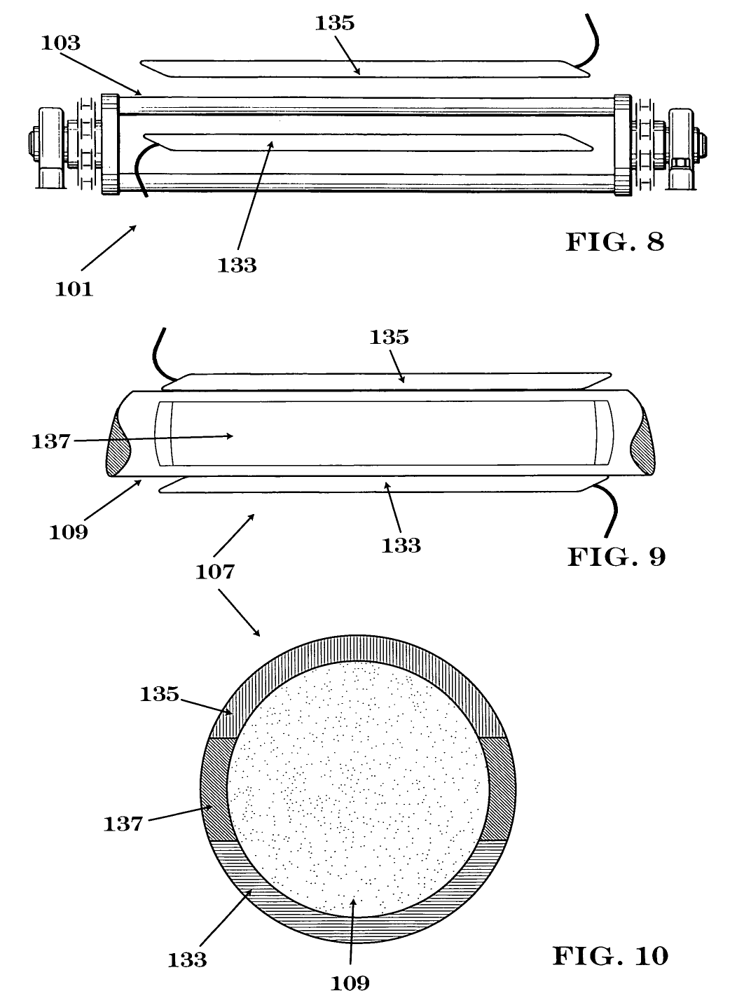

The fifth preferred embodiment uses PEF energy for the antimicrobial treatment of solid foodstuff. Referring now to FIG. 8, the fifth preferred embodiment is a conveyor IAMD 101 that has one or more anodes 133 and cathodes 135 oppositely mounted above, below, or surrounding conveyor 103. Foodstuff traveling along conveyor 103 is subjected to a PEF emanated by anodes 133 and cathodes 135. Preferably, conveyor 103 may be constructed of a nonmagnetic material such as plastic, or otherwise designed in a way that minimizes interference with the PEF, to ensure an effective treatment of the foodstuff with the PEF while the foodstuff is traveling along conveyor 103.

The fifth preferred embodiment utilizes the fact that solid foodstuffs contain high quantities of moisture, usually between about 10 and 96 percent, which represents and good conductive medium for a PEF. Microbes require moisture to thrive, and the fifth preferred embodiment uses such moisture to inactive the microbes using PEF. Preferably, the air surrounding conveyor 103 is ionized, or ionized water vapor is injected into conveyor IAMD 103 such that it envelops conveyor 103, in order to provide a uniform medium through which the PEF can travel to the solid foodstuff.

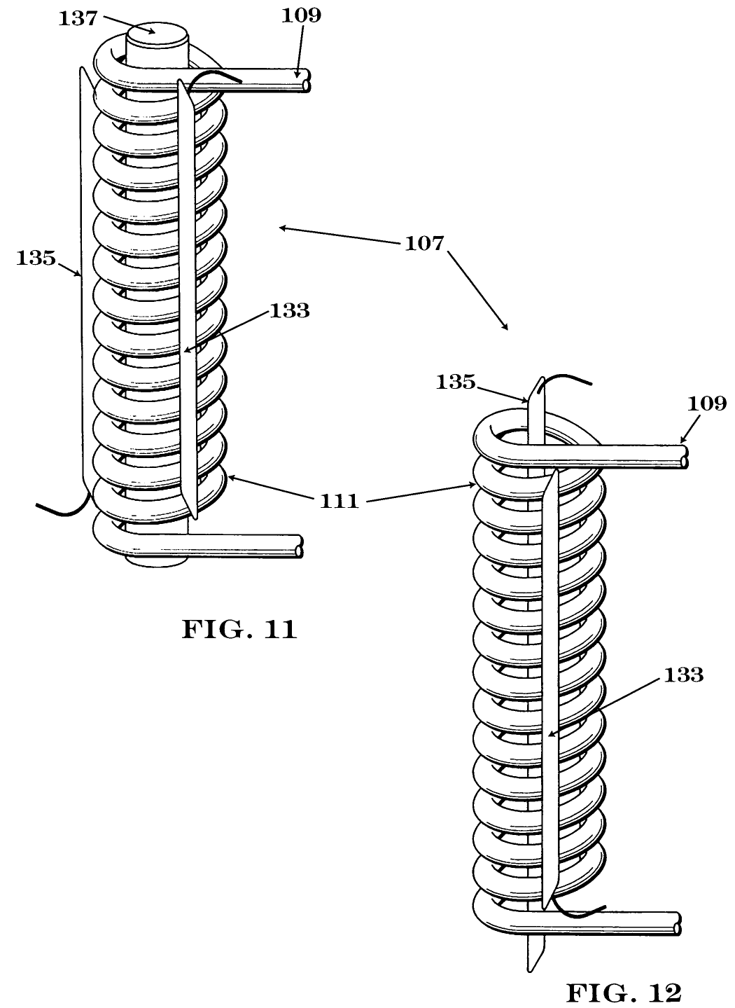

The sixth preferred embodiment uses PEF energy for the antimicrobial treatment of semisolid and liquid foodstuff. Referring now to FIG. 11, the sixth preferred embodiment is a conduit IAMD 107 that consists of one or more anodes 133 and cathodes 135 oppositely arranged on the outside of conduit 109 and separated by an insulator 137. The positions of anode 133 and cathode 135 are interchangeable and thus may be referred to interchangeably as electrodes. A pulsed charge is applied across anode 133 and cathode 135 to create a pulsed electrical field within conduit 109 in a direction perpendicular to the flow of food.

Referring now to FIG. 10, anode 133, cathode 135, and insulator 137 form the smooth, round interior surface of conduit 109. This embodiment allows full laminar flow through conduit 109 during PEF treatment. Furthermore, because the electrodes are in contact with the foodstuff moving through the conduit 109, this preferred configuration gives maximum energy efficiency as compared with configurations in which anodes 133 and cathodes 135 are outside conduit 109. This configuration may be used with both straight and spiraled conduit.

In addition to straight conduit applications, the sixth preferred embodiment may also be used where conduit 109 is spiraled or coiled. Referring now to FIGS. 11 and 12, conduit IAMD 107 consists of one or more anodes 133 and cathodes 135 positioned on the outside area of coil 111. Optionally, as showing in FIG. 12, one or more cathodes 135 may be positioned in the interior of coil 111. A pulsed charge is applied across anode 133 and cathode 135 to create a pulsed electrical field focused on a small portion of coil 111 in a direction substantially perpendicular to the flow of foodstuff. Preferably, the electrodes and insulator form at least a portion of the interior surface of conduit 109 as shown in FIG. 10.

As semisolid or liquid foodstuff flows through the conduit IAMD 107 of the sixth preferred embodiment, voltage is applied to anode 133, which creates an electric field between anode 133 and cathode 135. Preferably, the voltage applied is between about 1905 kV and 7620 kV, which applies an electric field across 1.5 inch (3.81 cm) wide conduit of between about 500 kV/cm and 2000 kV/cm for a time period of between 10 nanoseconds and 100 milliseconds, depending on the type of semisolid or liquid being treated. Foodstuff preferably has a residency time in the sixth preferred embodiment of one to three seconds, during which foodstuff is subjected to between 50 and 10,000 pulses.

### Magnetic Field Energy

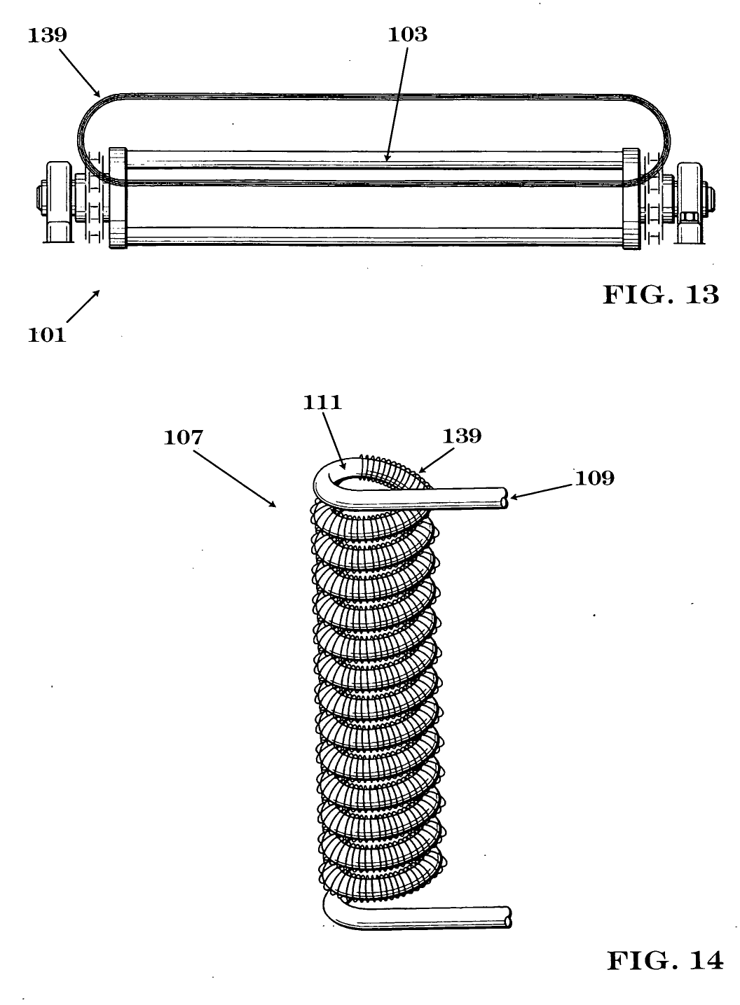

The seventh preferred embodiment uses magnetic field energy for the antimicrobial treatment of solid foodstuff. Referring now to FIG. 13, the seventh preferred embodiment is a conveyor IAMD 101 that has a magnetic coil 139 made of copper or other magnetic material mounted substantially around conveyor 103 in a direction substantially perpendicular to the direction of travel of conveyor 103. Conveyor 103 is preferentially made from nonmetallic material. As foodstuff travels along conveyor 103, magnetic coil 139 produces a magnetic field to which the foodstuff is subjected.

The eighth preferred embodiment uses magnetic field energy for the antimicrobial treatment of semisolid or liquid foodstuff. Referring now to FIG. 14, the eighth preferred embodiment is a conduit IAMD 107 that has a magnetic coil 139 made of copper or other magnetic material that surrounds conduit 109. Conduit 109 is preferentially made from a nonmetallic material such as plastic or glass. As foodstuff travels through conduit 109, magnetic coil 139 produces a magnetic field to which the foodstuff is subjected. If conduit 109 is straight pipe, then the magnetic coil 139 is arranged substantially perpendicular thereto. If conduit 109 is spiraled to form coil 111, magnetic coil 139 may surround the conduit 109 as shown or magnetic coil 139 may surround the entire coil 111 (not shown). Also, magnetic coil 139 may be spiraled through the interior of coil 111 (not shown).

In the seventh and eighth magnetic embodiments, a constant magnetic field of between about 1 tesla (T) to 20 T is applied to foodstuff, which disrupts the internal charges of ions within microbes and thus deactivates such microbes.

Persons having ordinary skill in the art will recognize that the modularity of the various preferred embodiments makes them well suited for use in multiple locations throughout a food processing system. Oftentimes, food processing systems involve repeated heating and cooling of food for various purposes, i.e., pasteurization, cooking, dethawing, etc. Several times during processing, food may pass through temperatures ranging from −2° C. to 55° C., which are conducive to microbial growth. Upon exiting such temperature ranges, particularly on the low side of such ranges, it is advantageous to use the invention to remove microbes from the foodstuff. The various embodiments of the invention may be used at virtually any temperature, but are preferentially used between about −25° C. and 80° C. This range is larger than the microbial growth range, which demonstrates that it may be advantageous to administer an antimicrobial treatment in a temperature environment in which the microbes are inactive and microbial growth is virtually zero.

In the preferred embodiments, conveyor IAMD 101 is scalable to treat solid foodstuff traveling on conveyor 103 at rates up to about 150 lb/min simply by altering the amount of power applied to the energy system installed in conveyor IAMD 101, if desired. By increasing the width and/or speed of conveyor 103 and the power capable of being applied to the energy system installed in conveyor IAMD 101, persons skilled in the art will recognize that conveyor IAMD 101 is scalable to handle foodstuffs traveling at far greater rates than 150 lb/min.

Likewise, in the preferred embodiments, conduit IAMD 107 is scalable to treat semisolid/liquid foodstuff traveling at rates up to 150 gal/min simply by altering the amount of power applied to the energy system installed in conduit IAMD 107, if desired. By increasing the diameter of conduit 109 and the power capable of being applied to the energy system installed in conduit IAMD 107, persons skilled in the art will recognize that conduit IAMD 107 is scalable to handle foodstuffs traveling at far greater rates than 150 gal/min.

### EXAMPLE

This example is intended to illustrate the flexibility with which the several embodiments of the invention may be deployed in a typical food transport system, and is not intended to limit the scope of the invention to the precise steps or order which follow. In a typical industrial food processing plant that utilizes meat emulsions, large chunks of meat enter the facility and are butchered to remove choice cuts of meat. The butchering stage typically takes place in a cool but not cold environment in which microbes may nonetheless grow, and butchering exposes additional surface area of the meat to microbes. Lesser choice cuts of meat are placed on a conveyor system to transport the meat cuts to a different area of the processing plant for grinding. Before reaching the grinder, the meat cuts are directed along the conveyor system through a first IAMD that treats against microbe contamination, for example, the seventh preferred embodiment, which subjects the meat cuts to an effective magnetic energy treatment as the meat cuts continue along the conveyor system. The conveyor system may additionally pass through a second antimicrobial treatment, as additional example, the first preferred embodiment, which subjects the meat cuts to a further effective treatment by ultrasound energy. As additional benefit to treatment against microbes, the ultrasound IAMD may additionally identify nonfood contamination in the meat cuts such as bone fragments left from butchering. Of those meat cuts that do not have contamination with bone fragments, they may continue along the conveyor system for grinding and mixing.

Once the meat is ground and mixed with emulsifiers, a meat emulsion, now semisolid in nature, moves through a food transport system in a conduit. At one or more times throughout processing toward ultimate product finishing, and at least just prior to packaging, the meat emulsion is pumped through a conduit IAMD. In this example, the conduit IAMD may deliver antimicrobial treatment according to the second preferred embodiment, i.e., effectively treating the meat emulsion with ultrasound. The conduit IAMD delivering ultrasound energy does not require additional physical handling of the meat emulsion, i.e., the conduit IAMD does not require any special batch processing.

As alternative example, the meat emulsion may be subjected to a conduit IAMD housing the seventh preferred embodiment, which applies a pulsed electric field to the meat emulsion. The PEF conduit IAMD may be located near a packaging unit, which wraps and seals the meat emulsion. The packaged meat emulsion, now a discrete unit, may now be placed on a conveyor that directs the packaged unit to further packaging, labeling, and shipping. At this time, just immediately prior to shipping or labeling, the packaged meat emulsion may be subjected to a third conveyor IAMD, housing, for example, the third preferred embodiment. The third preferred embodiment subjects the packaged meat emulsion to a combination of ruby and UV light that penetrates the packaging and eliminates microbes on and in the meat emulsion by virtue of the packaging process, if any. The packaged meat product may now be ready for shipping to a consumer.

The inventor has realized 7 log reductions in microbes on food by utilizing a combination of any or all four antimicrobial energy application methods disclosed herein. Additional benefits of using the several embodiments disclosed herein include implementation of one or more of these combinations without interrupting existing processing line speeds; the ability to treat packaged food; extending shelf life on packaged foodstuffs; detection of foreign substances such as plastic pieces or bone fragments in foodstuff prior to when the consumer ingests such foreign object; no substantial pressure or temperature increase due to treatment; the microbial reductions are unaffected by particulates in or clarity of liquids; and the flexibility to be adapted to virtually any preexisting food processing or other product automated or semi-automated operation.

While the inventor has described above what she believes to be the preferred embodiments of the invention, persons having ordinary skill in the art will recognize that other and additional changes may be made in conformance with the spirit of the invention and the inventor intends to claim all such changes as may fall within the scope of the invention.

I claim:

1. An antimicrobial treatment method comprising delivering an effective amount of ultrasound energy at frequency between about 855 kilohertz and 2 megahertz, thereby causing a reduction in live microbial content.

2. The antimicrobial treatment method of claim 1 further consisting of measuring the ultrasound energy after reflection to determine whether foreign or undesirable objects are present.

3. The antimicrobial treatment method of claim 1 wherein the effective amount of ultrasound energy is delivered during transport along a conveyor system.

4. The antimicrobial treatment method of claim 1 wherein the effective amount of ultrasound energy is delivered during transport through a conduit system.

5. An antimicrobial treatment device comprising:
at least one ultrasound driver capable of producing an effective amount of ultrasound energy at frequency between about 855 kilohertz and 2 megahertz;
at least one ultrasound transducer;
an article to be treated;
a conveyor system designed to transport the article,
a monitor capable of identifying whether nondesirable particles are present in the article and capable of signaling;
an inline means capable of receiving a signal from the monitor and removing or diverting the article from the conveyor system when nondesirable particles are detected in the article by the ultrasound transducer.

6. An antimicrobial treatment method comprising delivering an effective amount of light energy, said light energy consisting of the combination of ultraviolet light having wavelength between about 10 nanometers and 400 nanometers and ruby light having wavelength between about 560 nanometers and 1,000 nanometers, thereby causing a reduction in live microbial content.

7. The antimicrobial treatment method of claim 6 wherein the effective amount of light energy is delivered during transport along a conveyor system.

8. The antimicrobial treatment method of claim 6 wherein the effective amount of light energy is delivered during transport through a conduit system having transparent or semi-transparent qualities.

9. An antimicrobial treatment method comprising delivering an effective amount of pulsed electric field energy, said pulsed electric field energy having about 500 kilovolts per centimeter and 2,000 kilovolts per centimeter, a pulse length of between 10 nanosecond and 100 milliseconds, and between 50 and 10,000 individual pulses of electric field energy, thereby causing a reduction in live microbial content.

10. The antimicrobial treatment method of claim 9 wherein the effective amount of pulsed electric field energy is delivered during transport along a conveyor system.

11. The antimicrobial treatment method of claim 9 wherein the effective amount of pulsed electric field energy is delivered during transport through a conduit system.

12. An antimicrobial treatment method comprising delivering an effective amount of magnetic energy, said magnetic energy producing a magnetic field between about 1 tesla and 20 tesla, thereby causing a reduction in live microbial content.

13. The antimicrobial treatment method of claim 12 wherein the effective amount of magnetic energy is delivered during transport along a conveyor system.

14. The antimicrobial treatment method of claim 12 wherein the effective amount of magnetic energy is delivered during transport through a conduit system.
